(19) United States
(12) Patent Application Publication
(10) Pub. No.: US 2026/0271480 A1
(43) Pub. Date: Sep. 10, 2026

(54) PHOTOELECTRIC CONVERSION ELEMENT, PHOTOELECTRIC CONVERSION APPARATUS, MOVING BODY, AND BUILDING MATERIAL

(71) Applicant: CANON KABUSHIKI KAISHA, Tokyo (JP)

(72) Inventors: NANAMI KATO, Shizuoka (JP); KOHEI MAKISUMI, Shizuoka (JP); KUNIHIKO SEKIDO, Shizuoka (JP); YU YOSHIDA, Shizuoka (JP); TSUTOMU NISHIDA, Shizuoka (JP)

(21) Appl. No.: 19/657,776

(22) Filed: Apr. 24, 2026

Related U.S. Application Data
(63) Continuation of application No. PCT/JP2024/038182, filed on Oct. 25, 2024.

(30) Foreign Application Priority Data
Oct. 27, 2023 (JP) 2023-184750
Oct. 27, 2023 (JP) 2023-184756
Oct. 27, 2023 (JP) 2023-184761
Dec. 21, 2023 (JP) 2023-216294
Dec. 21, 2023 (JP) 2023-216296
Dec. 21, 2023 (JP) 2023-216299
Feb. 16, 2024 (JP) 2024-022244
Feb. 16, 2024 (JP) 2024-022246
Feb. 16, 2024 (JP) 2024-022251
May 28, 2024 (JP) 2024-086011
Oct. 23, 2024 (JP) 2024-186513

(51) Int. Cl. H10K 30/86; H02S 20/23; H02S 20/30; H10K 30/20; H10K 30/30; H10K 30/85; H10K 85/10; H10K 85/30; H10K 85/50

(52) U.S. Cl. CPC H10K 30/86; H10K 30/20; H10K 30/30; H10K 30/85; H10K 85/141; H10K 85/311; H10K 85/50; H02S 20/23; H02S 20/30

(57) ABSTRACT

A photoelectric conversion element having an improved short-circuit current density. The photoelectric conversion element is a photoelectric conversion element including a first electrode, a second electrode, and a photoelectric conversion layer arranged between the first electrode and the second electrode. The photoelectric conversion layer contains a crystal having a perovskite structure. The photoelectric conversion element includes, between the photoelectric conversion layer and the first electrode, a charge-transporting layer containing an amorphous body of a cyclic conjugated compound in which a plurality of pyrrole rings are bonded by conjugated bonds. In an X-ray diffraction spectrum of the charge-transporting layer using a CuKα ray, no diffraction peak having a half-width of 0.5° or less is present in a range of a Bragg angle 2θ of 6.5 to 8.5°.

### CROSS-REFERENCE TO RELATED APPLICATIONS

This application is a Continuation of International Patent Application No. PCT/JP2024/038182, filed Oct. 25, 2024, which claims the benefit of Japanese Patent Application No. 2023-184761, filed Oct. 27, 2023, Japanese Patent Application No. 2023-184756, filed Oct. 27, 2023, Japanese Patent Application No. 2023-184750, filed Oct. 27, 2023, Japanese Patent Application No. 2023-216294, filed Dec. 21, 2023, Japanese Patent Application No. 2023-216296, filed Dec. 21, 2023, Japanese Patent Application No. 2023-216299, filed Dec. 21, 2023, Japanese Patent Application No. 2024-022244, filed Feb. 16, 2024, Japanese Patent Application No. 2024-022251, filed Feb. 16, 2024, Japanese Patent Application No. 2024-022246, filed Feb. 16, 2024, Japanese Patent Application No. 2024-086011, filed May 28, 2024, and Japanese Patent Application No. 2024-186513, filed Oct. 23, 2024, all of which are hereby incorporated by reference herein in their entirety.

### BACKGROUND

### Field of the Technology

The present disclosure relates to a photoelectric conversion element, a photoelectric conversion apparatus, a moving body, and a building material.

### Description of the Related Art

In order to solve a disadvantage of the depletion of fossil energy and a global environmental disadvantage caused by the use of the fossil energy, investigations on a renewable and clean alternative energy source, such as solar energy, wind power, or water power, have been actively performed. In particular, an interest in a solar cell that directly changes sunlight into electrical energy has been increasing. The term “solar cell” as used herein means a battery that generates a current-voltage through utilization of a photovoltaic effect in which light energy is absorbed from sunlight to generate an electron and a hole.

Currently, an n-p diode-type silicon (Si) single crystal-based solar cell having a light energy conversion efficiency of more than 20% is widely known, and is actually used in solar power generation. However, the solar cell requires a high temperature treatment step and the price of a material itself is high, and hence there is a disadvantage in that the cost per unit electric power is high. In addition, there is a disadvantage with its supply property in terms of a silicon resource.

Meanwhile, a solar cell using an organic material (hereinafter also referred to as “organic solar cell”) does not require the high temperature treatment step, and can be produced in a so-called roll-to-roll system using a sheet-shaped substrate, and hence a cost reduction can be expected. However, further improvements in power generation efficiency and durability have been desired for practical use of the organic solar cell. In particular, the development of a perovskite solar cell including a crystal having a perovskite structure as a photoelectric conversion layer toward the practical use of the solar cell has been advanced because the cell is excellent in photoelectric conversion property. For example, Japanese Patent Laid-Open No. 2022-168820 discloses that a layer containing a phthalocyanine compound is formed between a hole-transporting layer (hereinafter also referred to as “charge-transporting layer”) and a perovskite to improve photoelectric conversion efficiency.

According to investigations made by the inventors, a further improvement in conversion efficiency toward practical use has been required to be achieved in the above-mentioned related art, and for that purpose, an improvement in short-circuit current density has been required.

### SUMMARY

The present disclosure is directed to providing a photoelectric conversion element having an improved short-circuit current density. The present disclosure is also directed to providing a photoelectric conversion apparatus, a moving body, and a building material each including the above-mentioned photoelectric conversion element.

The above-mentioned provision is achieved by the present disclosure described below. That is, the present disclosure is directed to a photoelectric conversion element including: a first electrode; a second electrode; and a photoelectric conversion layer arranged between the first electrode and the second electrode, the photoelectric conversion layer containing a crystal having a perovskite structure, wherein the photoelectric conversion element includes, between the photoelectric conversion layer and the first electrode, a charge-transporting layer containing an amorphous body of a cyclic conjugated compound in which a plurality of pyrrole rings are bonded by conjugated bonds, and wherein, in an X-ray diffraction spectrum of the charge-transporting layer using a CuKα ray, no diffraction peak having a half-width of 0.5° or less is present in a range of a Bragg angle 2θ of 6.5 to 8.5°.

The present disclosure is also directed to a photoelectric conversion apparatus including the above-mentioned photoelectric conversion element.

The present disclosure is also directed to a moving body including the above-mentioned photoelectric conversion element.

The present disclosure is also directed to a building material including the above-mentioned photoelectric conversion element.

Features of the present disclosure will become apparent from the following description of embodiments with reference to the attached drawings.

### BRIEF DESCRIPTION OF THE DRAWINGS

FIG. 1A is a schematic sectional view in a thickness direction of an example of a photoelectric conversion element of the present disclosure.

FIG. 1B is a schematic sectional view in a thickness direction of another example of the photoelectric conversion element of the present disclosure.

FIG. 2 is a schematic sectional view in a thickness direction of still another example of the photoelectric conversion element of the present disclosure.

FIG. 3 is a perspective view for schematically illustrating an example of a moving body including the photoelectric conversion element of the present disclosure.

FIG. 4 is a perspective view for schematically illustrating an example of a building material including the photoelectric conversion element of the present disclosure.

### DESCRIPTION OF THE EMBODIMENTS

### One Embodiment

One embodiment of the present disclosure is directed to a photoelectric conversion element.

A photoelectric conversion element of the present disclosure is a photoelectric conversion element including: a first electrode; a second electrode; and a photoelectric conversion layer arranged between the first electrode and the second electrode, the photoelectric conversion layer containing a crystal having a perovskite structure, wherein the photoelectric conversion element includes, between the photoelectric conversion layer and the first electrode, a charge-transporting layer containing an amorphous body of a cyclic conjugated compound in which a plurality of pyrrole rings are bonded by conjugated bonds, and wherein, in an X-ray diffraction spectrum of the charge-transporting layer using a CuKα ray, no diffraction peak having a half-width of 0.5° or less is present in a range of a Bragg angle 2θ of 6.5 to 8.5°.

The inventors have found that a photoelectric conversion element excellent in short-circuit current density can be obtained by incorporating the above-mentioned charge-transporting layer. Details of the reason why an excellent short-circuit current density is obtained in the present disclosure are not clear, but the inventors have presumed the mechanism to be as described below.

According to the prior-art literature, submicron unevenness occurs on a surface when a photoelectric conversion layer contains a crystal having a perovskite structure. It has been presumed that interfacial joining with an electrode is stabilized by filling a recess of the unevenness with a pigment particle formed of a phthalocyanine compound, which is a kind of cyclic compound in which a plurality of pyrrole rings are bonded by conjugated bonds, and hence high photoelectric conversion efficiency can be obtained. However, it has been found that this alone is insufficient to obtain a higher short-circuit current density.

Accordingly, the inventors have presumed that reduction in crystallinity of the cyclic conjugated compound in which a plurality of pyrrole rings are bonded by conjugated bonds, that is, use of an amorphous body contributes to improvement in short-circuit current density.

Specifically, in an X-ray diffraction spectrum of the charge-transporting layer using a CuKα ray (λ=1.5418 Å), the absence of a diffraction peak having a half-width of 0.5° or less (i.e., no diffraction peak is present, or a diffraction peak having a half-width of more than 0.5° is present) in the range of a Bragg angle 2θ of 6.5 to 8.5° enables the improvement in short-circuit current density.

The absence of a diffraction peak having a half-width of 0.5° or less in the range of a Bragg angle 2θ of 6.5 to 8.5° indicates that the cyclic conjugated compound is an amorphous body. The amorphous body includes not only a completely non-crystalline body but also a mixture of a crystalline body and an amorphous body.

The amorphous body of the cyclic conjugated compound generates fewer photoexcitons as compared to a compound including more crystalline bodies, and is hence capable of reducing trapping of holes for hole transportation. As a result, it is presumed that the improvement in short-circuit current density is achieved. The reason why the amorphous body generates fewer photoexcitons is presumed to be as described below. Molecules of the cyclic conjugated compound are not aligned, and hence an intermolecular distance is large and the overlap of π-conjugated electron clouds is weak. Thus, the photoexcitons are less separated to generate charge for charge transportation, and are more recombined.

In addition, the reason why attention has been paid to the scanning range of a Bragg angle 2θ of 6.5 to 8.5° is that it is assumed that characteristics of the amorphous body of the cyclic conjugated compound are further exhibited in this range because this scanning range corresponds to a larger intermolecular distance of the cyclic conjugated compound (an intermolecular distance of about 10 to 12 Å assuming from Bragg's equation) than that of a higher scanning range of a Bragg angle 2θ.

Based on the mechanism described above, the effects of the present disclosure can be achieved.

The present disclosure is described in detail below by way of preferred embodiments. The present disclosure is not limited to the following embodiments, and the following embodiments, which are appropriately changed, modified, and the like based on the ordinary knowledge of a person skilled in the art without departing from the gist of the present disclosure, are also encompassed within the scope of the present disclosure.

The term “layer” as used herein means not only a layer having a clear boundary or a layer having a flat thin film shape but also a layer having a concentration gradient in which the concentration of a constituent element gradually changes, or a layer that may form a complicatedly intricate structure together with another layer.

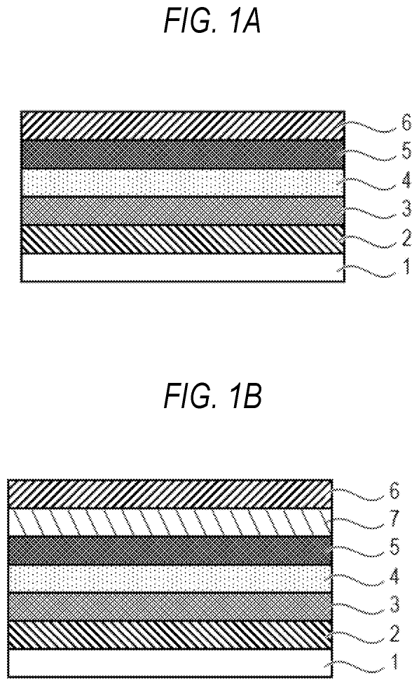

FIG. 1A is a sectional view for schematically illustrating the configuration of a photoelectric conversion element according to one embodiment of the present disclosure. The photoelectric conversion element includes a substrate 1, and a second electrode 2, an electron-transporting layer 3, a photoelectric conversion layer 4, a charge-transporting layer 5, and a first electrode 6 arranged thereon. One of the first electrode 6 and the second electrode 2 is an anode, and the other is a cathode. A current can be extracted by connecting the first electrode 6 and the second electrode 2 with an external circuit.

The photoelectric conversion layer 4 is excited by light that has entered the layer through the substrate 1, the second electrode 2, and the electron-transporting layer 3, or the first electrode 6 and the charge-transporting layer 5 to generate an electron or a hole. That is, the photoelectric conversion layer 4 generates a current between the first electrode 6 and the second electrode 2. The electron-transporting layer 3 is a layer arranged between the photoelectric conversion layer 4 and the second electrode 2, or between the second electrode 2 and the first electrode 6, and may be omitted in some cases. A form in which the plurality of electron-transporting layers 3 and photoelectric conversion layers 4 are laminated may be adopted. Such form may also be referred to as “tandem structure.”

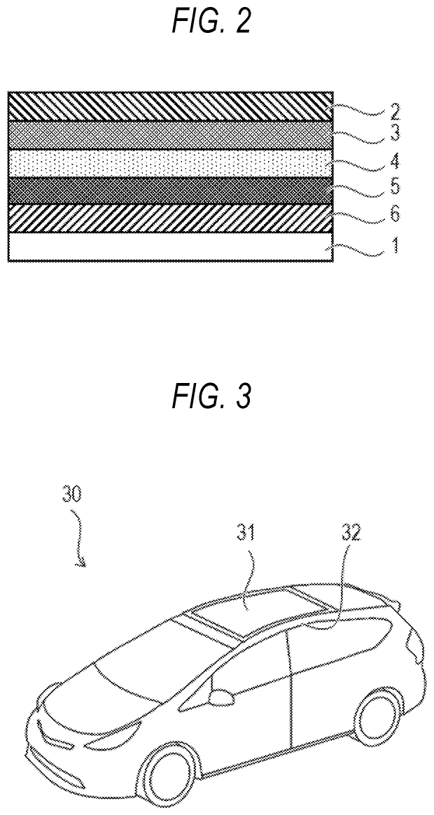

In addition, a configuration in which the first electrode 6, the charge-transporting layer 5, the photoelectric conversion layer 4, the electron-transporting layer 3, and the second electrode 2 are arranged on the substrate 1 may be adopted (FIG. 2).

The respective members are described below.

### [Photoelectric Conversion Element]

The photoelectric conversion element of the present disclosure is characterized by including: the first electrode 6; the second electrode 2; and the photoelectric conversion layer 4 arranged between the first electrode 6 and the second electrode 2, the photoelectric conversion layer containing the crystal having a perovskite structure, and including the charge-transporting layer 5 between the photoelectric conversion layer 4 and the first electrode 6. In addition, in order to improve the photoelectric conversion efficiency, a tandem type in which the photoelectric conversion elements are laminated may be adopted. The kind of the photoelectric conversion element to be laminated is not limited, and for example, a silicon solar cell or a CIGS solar cell may be adopted in addition to a perovskite solar cell using a crystal having a perovskite structure in the photoelectric conversion layer 4.

Examples of a method of forming each of the layers including the photoelectric conversion layer and charge-transporting layer of the present disclosure include a coating method and a vapor deposition method. Examples of the coating method include dip coating, spin coating, spray coating, ink jet coating, meniscus coating, screen coating, roll coating, die coating, blade coating, curtain coating, and wire bar coating. The coating method is a method including preparing a coating liquid for each layer to be described later, applying the liquid in the desired order of layers, and drying the liquid. A desired method may be selected as such forming method in accordance with the respective layers.

The respective layers are described below.

### [Substrate]

The photoelectric conversion element of the present disclosure may include the substrate 1, and examples thereof include a transparent glass substrate made of soda-lime glass or alkali-free glass, a ceramic substrate, and a transparent plastic substrate. In each of FIG. 1A and FIG. 1B, when light is taken in from the first electrode 6 side, an opaque material may be used as the substrate 1. In FIG. 2, when light is taken in from the second electrode 2 side, the substrate 1 is formed of a transparent material.

### [Electrode]

A material for the first electrode 6 or the second electrode 2 is not particularly limited, and a material that has hitherto been known may be used. Examples thereof include: metals, such as gold, silver, titanium, and copper; sodium; a sodium-potassium alloy; lithium; magnesium; carbon; a carbon nanotube; aluminum; a magnesium-silver mixture; a magnesium-indium mixture; an aluminum-lithium alloy; an Al/Al₂O₃ mixture; and an Al/LiF mixture. Examples of a transparent electrode material include: conductive transparent materials, such as CuI, indium tin oxide (ITO), SnO₂, aluminum zinc oxide (AZO), indium zinc oxide (IZO), gallium zinc oxide (GZO), fluorine-doped tin oxide (FTO), and antimony-doped tin oxide (ATO); and conductive transparent polymers. Those materials may be used alone or in combination thereof.

At least one electrode of the first electrode 6 or the second electrode 2 on a light incident side is a transparent electrode, and the other may be a transparent electrode or may also serve as a reflective layer formed of a light reflective material, or may be a transparent electrode including a reflective layer on a side opposite to the light incident side. In the photoelectric conversion element, it is preferred that the first electrode 6 be a positive electrode. When the first electrode 6 is on the light incident side, the second electrode 2 and the substrate 1 may be a transparent electrode and a reflective layer, respectively. The transparent electrode may be a patterned electrode.

### [Photoelectric Conversion Layer]

The photoelectric conversion layer 4 contains the crystal having a perovskite structure. The crystal having a perovskite structure to be used in the present disclosure is preferably represented by the following general formula [1].

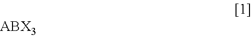

In the general formula [1], A represents a monovalent cation of an organic molecule or a metal atom, B represents a divalent metal cation, and X represents a monovalent halide anion.

A in the general formula [1] preferably represents CₚN_{q}Hᵣ (“p”, “q”, and “r” each represent a positive integer) in the case of, for example, the organic molecule. Specific examples thereof include methylammonium and formamidinium.

In addition, the metal atom is not particularly limited, and lithium, cesium, sodium, potassium, and rubidium are preferred. Those organic molecules or metal atoms may be used alone or in combination thereof.

When the cation A to be included is too large to fit in a crystal having a three-dimensional perovskite structure, a crystal having a two-dimensional perovskite structure, a crystal having a 2.5-dimensional perovskite structure with properties of both the two-dimensional and three-dimensional perovskite structures, a two-layer crystal having three-dimensional and two-dimensional perovskite structures, or a crystal having a mixed three-dimensional/two-dimensional perovskite structure is formed, and any of the structures functions as the photoelectric conversion layer 4. The two-layer crystal having three-dimensional and two-dimensional perovskite structures refers to a crystal in which the crystals having three-dimensional and two-dimensional perovskite structures are laminated as independent and separate layers. The crystal having a mixed three-dimensional/two-dimensional perovskite structure refers to a crystal having a structure in which both the regions or domains of crystals having two-dimensional or 2.5-dimensional layered and three-dimensional perovskite structures are mixed.

The crystal having a two-dimensional perovskite or 2.5-dimensional perovskite structure is preferably represented by each of the following general formula [2] to the following general formula [4].

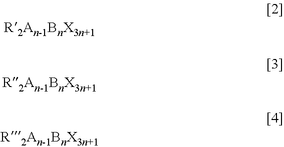

The general formula [2], the general formula [3], and the general formula [4] form perovskite structures of a Ruddlesden-Popper (RP) type, a Dion-Jacobson (DJ) type, and an Alternating cations in the interlayer (ACI) type, respectively.

R′, R″, and R′″ in the general formulae [2] to [4] each represent a cation of an organic molecule that may have a substituent or of a metal. Specifically, ethylammonium, propylammonium, n-butylammonium, n-hexylammonium, n-octylammonium, 1,6-hexanediammonium, isobutylammonium, 3-(nonafluoro-tert-butyloxy)propylamine, 1,3-propanediammonium, 1,5-pentamethylenediamine, octyldiammonium, 2,2-(ethylenedioxy)bis(ethylammonium), 5-aminovaleric acid, 4-tert-butylammonium, N,N′-dimethylethylene-1,2-diammonium, 2,2,3,3,3-pentafluoropropylammonium, guanidinium, propylammonium, propargylamine, alkylammonium, cyclohexylmethylammonium, pyrrolidinium, cyclohexylammonium, 4-fluorophenethylammonium, trifluoromethylbenzylammonium, pentafluorobenzylammonium, pentafluorophenylethylammonium, 4-methoxyphenethylammonium, imidazolium, pyridinium, 3-thiophenemethylammonium, 2-thiopheneethylammonium, 2-thiopheneformamidinium, 2-thiophenemethylammonium, 1-naphthylmethylammonium, 2-naphthylmethylammonium, phenethylammonium, phenylammonium, benzylammonium, 2,5-thiophenedimethylammonium, phenylpropylammonium, 1,4-phenylenedimethanamine, 3-phenyl-2-propen-1-ammonium, phenylbutylammonium, 4-tert-butylbenzylammonium, 3-(aminomethyl)piperidinium, and 4-(aminomethyl)piperidinium are preferred.

B in each of the general formula [1] to the general formula [4] represents a metal atom, and examples thereof include lead, tin, bismuth, zinc, titanium, antimony, nickel, iron, cobalt, silver, copper, gallium, germanium, magnesium, calcium, indium, aluminum, manganese, chromium, molybdenum, and europium. Of those, lead, tin, and bismuth are preferred from the viewpoint of the overlap of electron orbitals. Those metal atoms may be used alone or in combination thereof.

X in the general formula [1] to the general formula [4] represents a halogen atom or the like, and examples thereof include chlorine, bromine, iodine, sulfur, and selenium. Those halogen atoms may be used alone or in combination thereof. Of those, a halogen atom is preferred because, when the halogen atom is incorporated into the structure, the above-mentioned crystal having a perovskite structure easily becomes soluble in an organic solvent, and hence its application to an inexpensive printing method or the like is enabled. Further, iodine is more preferred because the energy bandgap of the crystal having a perovskite structure narrows.

Specifically, as three-dimensional perovskite, two-dimensional perovskite, and mixed three-dimensional/two-dimensional perovskite, MAPbI₃, FAPbCl₃, FAPbI₃, MAPbIₓBr₃₋ₓ, MAPbIₓCl₃₋ₓ, Cs_{0.05}(MA_{0.17}FA_{0.83})_{0.95}Pb(I_{0.83}Br_{0.17})₃, Cs_{0.05}FA_{0.88}MA_{0.07}PbI_{2.55}Br_{0.44}, (FAPbI₃)_{0.95}(MAPbBr₃)_{0.05}, (FAPbI₃)_{0.85}(MAPbBr₃)_{0.15}, CsPbI₃, CsPbBr₃, Csₓ(MA)₁₋ₓPbI₃, Csₓ(FA)₁₋ₓPbI₃, MAₓ(FA)₁₋ₓPbI₃, MA_{0.17}FA_{0.83}Pb(I_{0.83}Br_{0.17})₃, Cs_{0.15}FA_{0.85}PbI_{2.55}Br_{0.45}, Cs_{0.05}FA_{0.88}MA_{0.07}PbI_{2.56}Br_{0.44}, (PEA)₂(MA)₂Pb₃I₁₀, (PTA)₂(MA)₄Pb₅I₁₆, (PEA)₂(MA)₄Pb₅I₁₆, (ThMA)₂(MA)₂Pb₃I₁₀, (3BBA)₂(MA)₂Pb₃I₁₀, (ThMA)₂(FA)₄Pb₅I₁₆, (4FPEA)₂(FA_{0.3}MA_{0.7})₄Pb₅I₁₆, (PDMA)FA₂Pb₃I₁₀, (3AMPY)(MA)₃Pb₄I₁₃, (PDMA)MA₅Pb₆I₁₉, (PDMA)MA₃Pb₄I₁₃, (TTDMA)MA₃Pb₄I₁₃, (TTDMA)MA₄Pb₅I₁₆, (BA_{0.9}PEA_{0.1})₂MA₄Pb₅I₁₆, (BA_{0.9}PEA_{0.1})₂MA₃Pb₄I₁₃, (4FPEA)₂MA₃Pb₄I₁₃, (4FPEA)₂MA₄Pb₅I₁₆, (BA)₂MA₂Pb₃I₁₀, (BA)₂MA₃Pb₄I₁₃, (TEA)₂MA₂Pb₃I₁₀, (BA)₂MA₄Pb₅I₁₆, CsSnBr₃, CsSnI₃, FA_{0.75}MA_{0.25}Sn_{0.95}Ge_{0.05}I₃, FAMASnGeI₃, FASnBr₃, FASnI₃, MA₂Sn₃I₈, MASnBr₃, MASnGeI₃, and MASnI₃ are preferred.

The A site, B site, or X site of each of the general formulae may be adjusted to be deficient or excessive in accordance with purposes, and the combinations of x1 to x5 may be changed in accordance with purposes. The combinations of x1 to x5 are, for example, as shown in Table 1. Particularly preferred ranges are 0.03≤x1≤0.10, 0.80≤x2≤0.96, 0.95≤x3≤1.05, 0.80≤x4≤0.96, and 2.95≤x5≤3.05. MACI may be included as a material for forming a perovskite crystal.

TABLE 1

| x1 | x2 | 1-x2 | x3 | x4 | 1-x4 | x5 |
|---|---|---|---|---|---|---|
| 0.05 | 0.83 | 0.17 | 1.00 | 0.83 | 0.17 | 3.00 |
| 0.05 | 0.83 | 0.17 | 0.99 | 0.83 | 0.17 | 2.99 |
| 0.05 | 0.83 | 0.17 | 0.98 | 0.83 | 0.17 | 2.98 |
| 0.05 | 0.83 | 0.17 | 0.97 | 0.83 | 0.17 | 2.97 |
| 0.05 | 0.83 | 0.17 | 0.96 | 0.83 | 0.17 | 2.96 |
| 0.05 | 0.83 | 0.17 | 1.01 | 0.83 | 0.17 | 3.01 |
| 0.05 | 0.83 | 0.17 | 1.02 | 0.83 | 0.17 | 3.02 |
| 0.05 | 0.83 | 0.17 | 1.03 | 0.83 | 0.17 | 3.03 |
| 0.05 | 0.83 | 0.17 | 1.04 | 0.83 | 0.17 | 3.04 |
| 0.05 | 0.83 | 0.17 | 1.00 | 0.95 | 0.05 | 3.00 |
| 0.05 | 0.83 | 0.17 | 0.97 | 0.95 | 0.05 | 2.97 |
| 0.05 | 0.83 | 0.17 | 0.98 | 0.95 | 0.05 | 2.98 |
| 0.05 | 0.83 | 0.17 | 0.99 | 0.95 | 0.05 | 2.99 |
| 0.05 | 0.83 | 0.17 | 1.01 | 0.95 | 0.05 | 3.01 |
| 0.05 | 0.83 | 0.17 | 1.02 | 0.95 | 0.05 | 3.02 |
| 0.05 | 0.83 | 0.17 | 1.03 | 0.95 | 0.05 | 3.03 |

In the above-mentioned specific examples, “MA” represents methylammonium, “FA” represents formamidinium, “PEA” represents phenethyl ammonium, “PTA” represents phenyltriethylammonium, “ThMA” represents 2-thiophenemethylammonium, “3BBA” represents 3-bromobenzylammonium, “3AMPY” represents 3-(aminomethyl)pyridine, “PDMA” represents 1,4-phenylenedimethanammonium, “TTDMA” represents thieno[3,2-b]thiophene-2,5-diyldimethanammonium, “4FPEA” represents 4-fluorophenethylammonium, “BA” represents butylammonium, and “TEA” represents 2-thiophenethylammonium.

The above-mentioned crystal having a perovskite structure preferably has a cubic structure in which the metal atom B, the organic molecules A, and the halogen atom X are arranged on a body-centered position, the respective corners, and a face-centered position, respectively. The details are not clear, but it is assumed that, when such structure is present, the orientation of an octahedron in a crystal lattice can be easily changed, and hence the mobility of an electron in the crystal having a perovskite structure increases, and the photoelectric conversion efficiency of the photoelectric conversion element is improved.

The crystal having a perovskite structure to be used in the present disclosure is preferably a crystalline semiconductor. The term “crystalline semiconductor” means a semiconductor that enables measurement of an X-ray scattering intensity distribution to detect a scattering peak. When the crystal having a perovskite structure is the crystalline semiconductor, the mobility of the electron in the crystal having a perovskite structure increases, and the photoelectric conversion efficiency of the photoelectric conversion element is improved.

The thickness of the photoelectric conversion layer 4 according to the present disclosure is preferably 5 to 2,000 nm. When the thickness is 5 nm or more, light can be sufficiently absorbed, and when the thickness is 2,000 nm or less, the generated charge can be transported to the respective electrodes. A more preferred lower limit is 50 nm or more, a more preferred upper limit is 1,200 nm, a still more preferred lower limit is 100 nm, and a still more preferred upper limit is 1,000 nm.

### [Charge-Transporting Layer]

The photoelectric conversion element of the present disclosure includes, between the photoelectric conversion layer 4 and the first electrode 6, the charge-transporting layer 5 containing an amorphous body of a cyclic conjugated compound in which a plurality of pyrrole rings are bonded by conjugated bonds.

The cyclic conjugated compound in which a plurality of pyrrole rings are bonded by conjugated bonds to be used in the present disclosure is preferably a porphyrin compound or a phthalocyanine compound, more preferably a phthalocyanine compound from the viewpoint of the spreading of a π-electron cloud serving as the starting point of an interaction. In the photoelectric conversion element of the present disclosure, the phthalocyanine compound is preferably free of a central metal or preferably has Ga, Cu, Ti, Al, Si, Zn, V, Pb, Pt, Co, Sn, Mg, Fe, and Mn as a central metal. Specifically, gallium, aluminum, titanium, or silicon may be used as the central metal, and a gallium phthalocyanine compound may be more preferred.

Specific examples of the cyclic conjugated compound in which a plurality of pyrrole rings are bonded by conjugated bonds in the present disclosure are listed below.

Specific examples of the cyclic conjugated compound in which a plurality of pyrrole rings are bonded by conjugated bonds in the present disclosure include porphyrin derivatives, such as tetraphenylporphyrin, diphenylporphyrin, tetrapyridylporphyrin, copper porphyrin, copper tetraphenylporphyrin, copper octaethylporphyrin, cobalt tetraphenylporphyrin, octaethylporphyrin, chlorophenylporphyrin, methoxyphenylporphyrin, methylphenylporphyrin, zinc porphyrin, magnesium porphyrin, octabutoxyporphyrin, manganese chloroporphyrin, metal-free tetraazaporphyrin, copper tetraazaporphyrin, zinc tetraazaporphyrin, nickel tetraazaporphyrin, titanyl tetraazaporphyrin, tetraphenyltetraazaporphyrin, and octaphenyltetraazaporphyrin.

In addition, specific examples of the cyclic conjugated compound in which a plurality of pyrrole rings are bonded by conjugated bonds in the present disclosure include phthalocyanine derivatives, such as hydroxygallium phthalocyanine, chlorogallium phthalocyanine, copper phthalocyanine, zinc phthalocyanine, phthalocyanine, cobalt phthalocyanine, titanyl phthalocyanine, dichlorotin phthalocyanine, magnesium phthalocyanine, tin phthalocyanine, lead phthalocyanine, iron phthalocyanine, vanadyl phthalocyanine, chloroaluminum phthalocyanine, nickel phthalocyanine, dichlorosilicon phthalocyanine, indium chlorophthalocyanine, manganese phthalocyanine, chloroiron phthalocyanine, and platinum phthalocyanine. In the photoelectric conversion element of the present disclosure, the gallium phthalocyanine compound is preferably hydroxygallium phthalocyanine.

In addition, specific examples of the cyclic conjugated compound in which a plurality of pyrrole rings are bonded by conjugated bonds in the present disclosure include naphthalocyanine derivatives, such as naphthalocyanine, magnesium naphthalocyanine, copper naphthalocyanine, magnesium naphthalocyanine, cobalt naphthalocyanine, vanadyl naphthalocyanine, tin naphthalocyanine, and dichlorotin naphthalocyanine. In the present disclosure, the chemical structure of the cyclic conjugated compound in which a plurality of pyrrole rings are bonded by conjugated bonds or the like may be identified by, for example, nuclear magnetic resonance (NMR).

In the photoelectric conversion element of the present disclosure, the charge-transporting layer 5 preferably contains a resin. When the content of the cyclic conjugated compound in the charge-transporting layer 5 is set to 100 parts by mass, the content of the resin in the charge-transporting layer 5 is preferably 3 to 30 parts by mass, more preferably 5 to 20 parts by mass from the viewpoints of a film-forming property and a charge-transporting ability. The content of the resin may be quantified with a nuclear magnetic resonance (NMR) apparatus or a gas chromatography apparatus.

In addition, the molecular weight of the resin is preferably 10,000 or more. Further, the glass transition temperature (Tg) of the resin is preferably 95° C. or less. When the temperature falls within the range, the resin is easily brought into close contact with a charge-transporting material (charge-transporting particle), and hence a more effective charge distribution can be formed. The glass transition temperature may be determined with a differential scanning calorimeter (DSC).

Examples of the resin to be preferably used in the present disclosure include a polyester resin, a polycarbonate resin, a polyvinyl acetal resin, a polyvinyl butyral resin, an acrylic resin, a polyvinyl alcohol resin, a cellulose resin, a polystyrene resin, a polyvinyl acetate resin, and a polyvinyl chloride resin.

The charge-transporting layer may contain an aromatic ring compound containing a hydroxy group, and the content of the aromatic ring compound containing a hydroxy group in the charge-transporting layer is preferably 1 to 30 mass %, more preferably 5 to 20 mass % with respect to the content of the cyclic conjugated compound in which a plurality of pyrrole rings are bonded by covalent bonds from the viewpoints of the film-forming property and the charge-transporting ability.

An example of the aromatic ring compound containing a hydroxy group to be preferably used in the present disclosure is a calixarene compound.

Moreover, in the photoelectric conversion element of the present disclosure, in the X-ray diffraction spectrum of the charge-transporting layer 5 using a CuKα ray, no diffraction peak having a half-width of 0.5° or less is present in the range of a Bragg angle 2θ of 6.5 to 8.5°. That is, in the photoelectric conversion element of the present disclosure, in the range of the Bragg angle 2θ of 6.5 to 8.5° of the X-ray diffraction spectrum of the charge-transporting layer using a CuKα ray, a diffraction peak having a half-width of 0.6° or more may be present, and preferably, a peak having a half-width of 1.0° or more may be present. Those peaks indicate that the cyclic conjugated compound in which a plurality of pyrrole rings are bonded by conjugated bonds is an amorphous body. In addition, the amorphous body includes an amorphous body in which the ratio of crystals is so small that a peak is too broad to be detected.

In the photoelectric conversion element of the present disclosure, in the X-ray diffraction spectrum of the charge-transporting layer using a CuKα ray, peaks are preferably present in the ranges of a Bragg angle 2θ of 6.8 to 7.10 and 16.4 to 16.8°. In this way, the short-circuit current density of the photoelectric conversion element becomes more satisfactory.

Examples of a dispersion method for forming particles of the amorphous body of the cyclic conjugated compound in which a plurality of pyrrole rings are bonded by conjugated bonds include methods using a paint shaker, a sand mill, a ball mill, and a liquid collision-type high-speed disperser.

Of those, a sand mill forms particles of an amorphous body by rotation of a disc rotating in a mill and a shearing force by a medium such as glass beads serving as a grinding medium. The half-width of the peak changes because the crystallinity of an amorphous body changes depending on dispersion conditions, such as a dispersion time, an amount of beads, and the number of rotations of the disc at the time of the formation of particles. For example, the half-width tends to increase when the number of rotations of the disc is reduced to the extent that non-uniform dispersion does not occur.

Measurement of the X-ray diffraction spectrum of the charge-transporting layer 5 used in the present disclosure and recognition of the cyclic compound included therein in which a plurality of pyrrole rings are bonded by conjugated bonds or the like were performed by the following methods after layers above the charge-transporting layer 5 of the photoelectric conversion element of the present disclosure were removed with an organic solvent such as chloroform and a surface of the charge-transporting layer 5 was exposed.

### [Analysis of Amount of Compound]

The surface of the charge-transporting layer 5 was wiped with a cotton swab or the like to which a solvent had been applied, and was dissolved in deuterated sulfuric acid, and ¹H-NMR measurement (apparatus: AVANCE III 500, manufactured by BRUKER Corporation) was performed. In addition, the presence of the compound was determined by subjecting the component that had been stripped off to mass and structural analyses through GPC and MALDI-TOF-MS, IR, and gas chromatography.

In addition, the thickness of the layer was determined by cross-sectional SEM (apparatus: SmartSEM, manufactured by Carl Zeiss Co., Ltd.) after the photoelectric conversion element was cut and fixed to an inclined sample stage.

### MALDI-TOF-MS Analysis

The molecular weight of the compound was determined from an obtained peak top value under the following conditions.
Measuring instrument used: matrix-assisted laser desorption ionization time-of-flight mass spectrometer (MALDI-TOF MS) ultraflex manufactured by Bruker Daltonics K.K.
Acceleration voltage: 20 kV
Mode: Reflector
Molecular weight standard: Fullerene C60

### [X-ray Diffraction Measurement]

The X-ray diffraction spectrum of the exposed charge-transporting layer 5 was measured, and a value of a Bragg angle 2θ of a peak present in the range of a Bragg angle 2θ of 6.5 to 8.5° (described as “peak present in range of 20 of 6.5 to 8.5° ” in Table 2) and a half-width of the peak (described as “half-width” in Table 2) were determined.
Measuring instrument used: X-ray diffractometer RINT-TTRII manufactured by Rigaku Corporation
X-ray tube: Cu
X-ray wavelength: Kα1
Tube voltage: 50 KV
Tube current: 300 mA
Scanning method: 2θ-θ scan
Scanning speed: 4.0°/min
Sampling interval: 0.02°
Start angle 2θ: 3.0°
Stop angle 2θ: 30.0°
Goniometer: Rotor horizontal goniometer (TTR-2)
Filter: None
Detector: Scintillation counter
Incident monochromator: Used
Slit: Variable slit (Parallel beam method)
Counter monochromator: Not used
Divergence slit: Open
Divergence longitudinal limiting slit: 10.00 mm
Scattering slit: Open
Receiving slit: Open

### [Second Charge-Transporting Layer]

As illustrated in FIG. 1B, the photoelectric conversion element of the present disclosure preferably includes the second charge-transporting layer 7 between the charge-transporting layer 5 and the first electrode 6 from the viewpoint of the compatibility of a film of the charge-transporting layer. When the photoelectric conversion element includes the second charge-transporting layer 7, the charge-transporting layer (charge-transporting layer 5) containing a phthalocyanine crystal serves as a first charge-transporting layer.

A material for the second charge-transporting layer 7 is not particularly limited, and examples thereof include a spirofluorene compound, a triphenylamine compound, a chrysene compound, a pyrene compound, a phthalocyanine compound, a carbazole compound, a fluorene compound, a phenylcyclohexane compound, a benzidine compound, a phenoxazine compound, a phenylenediamine compound, a thiocyanate compound, and a thiophene compound. The compound particularly preferably has an aromatic ring from the viewpoint of the compatibility of a film interface, and Spiro-OMeTAD, PTAA, or a phthalocyanine compound is preferred.

In addition, the second charge-transporting layer may contain a dopant as an additive in order to improve the charge-transporting ability. Examples of a substance that may be used as the dopant include lithium compounds such as lithium bis(trifluoromethanesulfonyl)imide, cobalt compounds such as [tris(2-(1H-pyrazol-1-yl)-4-tert-butylpyridine)cobalt(III) tris(bis(trifluoromethylsulfonyl)imide)], boron compounds such as tetrakis(pentafluorophenyl)borate, molybdenum compounds such as tris[1-(methoxycarbonyl)-2-(trifluoromethyl)-ethane-1,2-dithiolene]molybdenum, organic compounds each having a tetracyanoquinodimethane skeleton such as 2,3,4,6-tetrafluoro-7,7,8,8-tetracyanoquinodimethane, and organic compounds each having a pyridine skeleton such as 4-tert-butylpyridine.

### [Electron-Transporting Layer]

In the photoelectric conversion element of the present disclosure, the electron-transporting layer 3 may be arranged between the second electrode 2 and the photoelectric conversion layer 4 as illustrated in FIG. 1A.

A material for the electron-transporting layer 3 is not particularly limited, and examples thereof include an N-type conductive polymer, an N-type low-molecular-weight organic semiconductor, an N-type metal oxide, an N-type metal sulfide, a halogenated alkali metal, an alkali metal, and a surfactant. Specific examples thereof include a cyano group-containing polyphenylene vinylene, a boron-containing polymer, bathocuproine, bathophenanthroline, hydroxyquinolinatoaluminum, an oxadiazole compound, a benzimidazole compound, a naphthalenetetracarboxylic acid compound, a fullerene compound, a perylene derivative, a phosphine oxide compound, a phosphine sulfide compound, a fluoro group-containing phthalocyanine, titanium oxide, zinc oxide, indium oxide, tin oxide, gallium oxide, tin sulfide, indium sulfide, and zinc sulfide.

A preferred lower limit of the thickness of the electron-transporting layer 3 is 1 nm, and a preferred upper limit thereof is 2,000 nm. When the thickness of the electron-transporting layer 3 is 1 nm or more, a hole can be sufficiently blocked, and when the thickness is 2,000 nm or less, the electron-transporting layer 3 is less liable to serve as resistance at the time of the electron transportation, and hence the photoelectric conversion efficiency increases. A more preferred lower limit of the thickness is 3 nm, a more preferred upper limit thereof is 1,000 nm, a still more preferred lower limit thereof is 5 nm, and a still more preferred upper limit thereof is 500 nm.

### Application Examples

Application examples of the present disclosure are directed to a photoelectric conversion apparatus, a moving body, and a building material.

### [Photoelectric Conversion Apparatus]

A photoelectric conversion apparatus of the present disclosure includes the photoelectric conversion element of the present disclosure. The photoelectric conversion apparatus may be formed by using a plurality of photoelectric conversion elements of the present disclosure. When a plurality of photoelectric conversion elements are connected, such photoelectric conversion apparatus may also be referred to as “photoelectric conversion cell” or “photoelectric conversion module.” In the photoelectric conversion element, elements having different absorption wavelengths may be laminated to increase an output voltage. In addition, the photoelectric conversion apparatus includes the photoelectric conversion element of the present disclosure and an inverter. The inverter may be a converter for converting a DC voltage to an AC voltage. The photoelectric conversion apparatus may include an electricity storage unit connected to the photoelectric conversion element. The electricity storage unit is not limited as long as the electricity storage unit can store electricity. Examples thereof include a secondary battery using lithium ions, an all-solid-state battery, and an electric double layer capacitor. In order to impart a function of, for example, maintaining or increasing the amount of incident light, a surface layer to which water or dirt is hard to adhere, or a function of collecting or guiding light may be added.

### [Moving Body]

A moving body of the present disclosure includes the photoelectric conversion element of the present disclosure. FIG. 3 is a perspective view for schematically illustrating a moving body including the photoelectric conversion element according to one embodiment of the present disclosure. A moving body 30 includes a photoelectric conversion element 31 of the present disclosure and a body 32 including the photoelectric conversion element 31. The photoelectric conversion element 31 is arranged on the position of the body 32 at which ambient light can be received. When the moving body 30 is an automobile, the photoelectric conversion element 31 may be arranged on a roof. Electric energy obtained by the photoelectric conversion element 31 may serve as the power of the moving body 30 or the power of any other electric equipment. Electric energy generated from the power of the moving body 30 may be used for the power of the photoelectric conversion element 31. When the moving body 30 is an automobile, friction energy generated with a brake may be converted into electric energy to be used for the control of the photoelectric conversion element 31.

The moving body 30 may be, for example, an automobile, a motorcycle, a railway vehicle, a ship, or a flying body including an artificial satellite, an airplane, and a drone. The configuration of the body 32 of the moving body 30 is not particularly limited, but is preferably formed of a material having high strength.

### [Building Material]

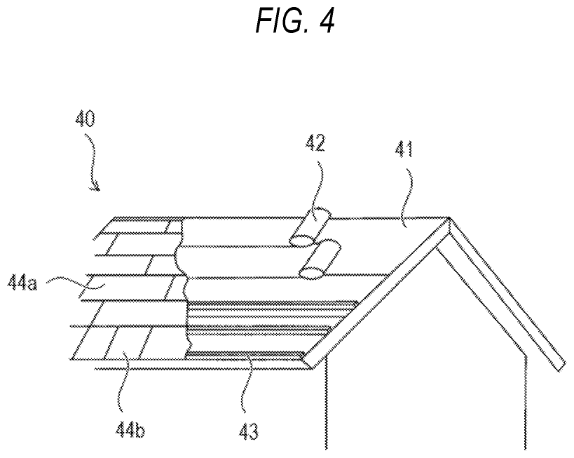

A building material of the present disclosure includes the photoelectric conversion element of the present disclosure. FIG. 4 is a perspective view for schematically illustrating a building material including the photoelectric conversion element according to one embodiment of the present disclosure. A building material 40 may be a roof of a building. The building material 40 of this embodiment includes a photoelectric conversion element 41 of the present disclosure, a protective member 42 for protecting the photoelectric conversion element 41, a heat dissipation member 43, and exteriors 44a and 44b.

The building material 40 of the present disclosure may include the heat dissipation member 43 having a thermal conductivity higher than that of the photoelectric conversion element 41. When the building material 40 is used for a roof or the like, the temperature of the photoelectric conversion element 41 may be increased by sunlight, and hence the photoelectric conversion efficiency may be reduced. The reduction of the photoelectric conversion efficiency can be suppressed by using the heat dissipation member 43. Examples of the heat dissipation member 43 include a metal, an alloy, a liquid metal, and a liquid resin.

In addition, the building material 40 of the present disclosure may include the exteriors 44a and 44b. The exterior 44a and the exterior 44b may show different colors, or may show the same color. The exterior 44a and the exterior 44b may be formed of the same member or may be formed of different members. A paint or a transparent substrate may be used as the exterior. An exterior having small light absorption and a high heat-shielding property is preferred.

In addition to the application examples described above, the following application examples are given: portable devices, such as a calculator, a sensor, and a small solar panel; wearable devices, such as a glasses-type terminal, a watch-type terminal, and a portable medical device; sheet structures supported by a plurality of frames, such as a tent, a plastic greenhouse, and a loading platform of a truck; and structures to be used by being fixed, such as a road surface panel, a floating panel, a building material utilizing the flexibility of a substrate, a wall-type building material, a glass-type building material, and a mega solar panel.

### [Method of Producing Photoelectric Conversion Element]

A method of producing a photoelectric conversion element of the present disclosure includes the steps of: forming a first electrode; forming a second electrode; and forming a photoelectric conversion layer containing a crystal having a perovskite structure between the first electrode and the second electrode. The respective steps of the production method are described below.

### (Step of Forming First Electrode and Step of Forming Second Electrode)

The method of producing a photoelectric conversion element of the present disclosure includes the steps of: forming the first electrode; and forming the second electrode. In the step of forming the first electrode and the step of forming the second electrode, an appropriate method may be selected in accordance with a material for the first electrode and a material for the second electrode, respectively. Examples of such method include, but are not limited to, a sputtering method, a vacuum vapor deposition method, a vapor phase growth method (CVD method), and a spray pyrolysis deposition method (SPD method). Materials for the first electrode and the second electrode are as described above. When one, or each of both, of the first electrode and the second electrode is a transparent electrode, the thickness of the transparent electrode is preferably 0.03 to 3 μm.

When a solar cell is produced, cutting processing may be performed for circuit formation between steps. Examples of the cutting processing include mechanical patterning and laser patterning.

### (Modularization Step)

An element formed up to the electrode may be sealed. Examples of a method for the sealing include sealing with a resin or sealing with a film. Examples of a material used for the sealing include a silazane, a silicone rubber, a resin having a siloxane skeleton, and glass.

In addition, hairline treatment may be performed on a surface of the sealed element from the viewpoint of the suppression of adhesion between elements occurring during winding in a roll-to-roll system.

### (Step of Forming Photoelectric Conversion Layer)

The step of forming the photoelectric conversion layer may include a step of applying a liquid containing the material for the photoelectric conversion layer described above. Examples of a method for the application include a spin coating method, a blade coating method, a slit die coating method, a screen printing method, a bar coater method, a casting method, a printing transfer method, a dip-drawing method, an ink jet method, a spray method, and a vacuum vapor deposition method. The method is appropriately selected therefrom in accordance with the properties of a photoelectric conversion layer to be produced, such as thickness control and orientation control.

Annealing treatment may be performed under reduced pressure or under an inert atmosphere (under a nitrogen or argon atmosphere) in order to remove a solvent or a dispersion medium from the applied liquid containing the material for the photoelectric conversion layer. The temperature of the annealing treatment is preferably 40 to 300° C., more preferably 50 to 150° C. The annealing treatment is preferably performed because materials for forming each layer may permeate each other at an interface between laminated layers to increase a contact area, and hence a short-circuit current can be increased.

### (Step of Forming Charge-Transporting Layer)

As a step of forming the charge-transporting layer, a method of applying a liquid containing the material for the charge-transporting layer described above is preferred. Examples of a method for the application include a spin coating method, a blade coating method, a slit die coating method, a screen printing method, a bar coater method, a casting method, a printing transfer method, a dip-drawing method, an ink jet method, a spray method, and a vacuum vapor deposition method.

In addition, examples of the step of forming the charge-transporting layer include: a method including arranging a charge-transporting particle on a surface of a photoelectric conversion layer, and then applying and drying a resin solution in which a resin is dissolved; a method including applying a resin solution in which a resin is dissolved on a surface of a photoelectric conversion layer, then arranging a charge-transporting particle, and then drying the resin solution; and a method including applying a solution in which a charge-transporting particle is dispersed in a resin solution in which a resin is dissolved onto a surface of a photoelectric conversion layer, and drying the solution.

### EXAMPLES

The present disclosure is described in more detail below by way of Examples and Comparative Examples. The present disclosure is by no means limited to the following Examples without departing from the gist thereof. In the description of the following Examples, “part(s)” is by mass unless otherwise specified.

(Process of Producing Particle 1 Containing Amorphous Body of Cyclic Compound in which Plurality of Pyrrole Rings are Bonded by Conjugated Bonds)

### Step (1)

Under a nitrogen flow atmosphere, 5.46 parts of orthophthalonitrile and 45 parts of α-chloronaphthalene were loaded into a reaction kettle. After that, the mixture was heated so that its temperature was increased to 30° C., followed by the maintenance of the temperature. Next, 3.75 parts of gallium trichloride was loaded into the mixture at the temperature (30° C.). The moisture concentration of the mixed liquid at the time of the loading was 150 ppm. After that, the temperature of the mixed liquid was increased to 200° C. Next, under a nitrogen flow atmosphere, the mixed liquid was subjected to a reaction at a temperature of 200° C. for 4.5 hours, and was then cooled. The product was filtered when its temperature reached 150° C. The resultant filter residue was subjected to dispersion washing with N,N-dimethylformamide at a temperature of 140° C. for 2 hours, and was then filtered. The resultant filter residue was washed with methanol, and was then dried to provide a chlorogallium phthalocyanine particle in a yield of 71 mass %.

### Step (2)

4.65 Parts of the chlorogallium phthalocyanine particle was dissolved in 139.5 parts of concentrated sulfuric acid at a temperature of 10° C., and the solution was dropped into 620 parts of ice water under stirring so that the particle was reprecipitated, followed by filtration with a filter press under reduced pressure. At this time, No. 5C (manufactured by Advantec Toyo Kaisha, Ltd.) was used as a filter. The resultant wet cake (filter residue) was subjected to dispersion washing with 2% ammonia water for 30 minutes, and was then filtered with the filter press. Next, the resultant wet cake (filter residue) was subjected to dispersion washing with ion-exchanged water, and then its filtration with the filter press was repeated three times. Finally, the filter residue was freeze-dried to provide a hydroxygallium phthalocyanine particle (hydrous hydroxygallium phthalocyanine particle) having a solid content of 23 mass % in a yield of 71 mass %. The hydroxygallium phthalocyanine particle was dried with a hyper-dry dryer (product name: HD-06R, frequency (oscillatory frequency): 2,455 MHz±15 MHz, manufactured by Biocon (Japan) Ltd.). Thus, a particle 1 having a water content of 1.0 mass % or less was obtained. It was recognized that, in the X-ray diffraction spectrum of the particle 1 using a CuKα ray, peaks were present in the ranges of a Bragg angle 2θ of 6.8 to 7.1° and 16.4 to 16.8°.

### (Production of Resin Solution 1)

1.0 Gram of polyvinyl butyral (product name: BM-2, manufactured by Sekisui Chemical Co., Ltd.) was dissolved in 19 g of 2-propanol by stirring for 24 hours to provide a resin solution 1.

### (Production of Resin Solution 2)

1.0 Gram of polyacrylic acid (molecular weight: 5,000, manufactured by FUJIFILM Wako Pure Chemical Corporation) was dissolved in 19 g of ethanol by stirring for 24 hours to provide a resin solution 2.

### Example 1

### [Formation of Electron-Transporting Layer]

A glass substrate with ITO was washed, and tin(II) oxide whose concentration was adjusted to 3 mass % was applied thereonto by spin coating. After that, the resultant was heated at 150° C. for 30 minutes to form an electron-transporting layer as a thin film having a thickness of 15 nm.

### [Formation of Photoelectric Conversion Layer]

22.4 Milligrams of methylammonium bromide, 172 mg of formamidinium iodide, and 576 mg of lead iodide were dissolved in 600 μL of N,N-dimethylformamide and 160 μL of dimethyl sulfoxide, and the solution was stirred for 1 hour (solution 1). Further, 389.72 mg of cesium iodide was dissolved in 1,000 μL of dimethyl sulfoxide, and the solution was stirred for 1 hour (solution 2). After that, 40 μL of the cesium iodide solution (solution 2) was added to the solution 1 to prepare a coating liquid for a photoelectric conversion layer. The coating liquid was applied onto the electron-transporting layer by spin coating to form a photoelectric conversion layer formed of Cs_{0.05}(FA_{0.83}MA_{0.17})_{0.96}Pb(I_{0.95}Br_{0.05})₃ and having a thickness of 500 nm.

### [Formation of Charge-Transporting Layer 1]

5.0 Grams of the particle 1 and 0.5 g of a calixarene compound (Exemplified Compound 1 described in Japanese Patent Laid-Open No. 2003-207913) were loaded into a container together with 560 g of 2-propanol and 580 g of zirconia beads, and dispersion treatment was performed with a sand mill (manufactured by Igarashi Machine Production Co., Ltd. (currently Aimex Co., Ltd.), disc diameter: 70 mm, number of discs: 5) for 30 hours by rotating discs at 300 rpm to prepare a coating liquid for a charge-transporting layer. The coating liquid for a charge-transporting layer was applied onto the photoelectric conversion layer by spin coating to form a charge-transporting layer having a thickness of 100 nm.

### [Introduction of Second Charge-Transporting Layer]

1.5 Grams of Spiro-OMeTAD serving as a material for a second charge-transporting layer was dissolved in 2.2 g of chlorobenzene. 36 Microliters of an acetonitrile solution obtained by dissolving 0.2 g of lithium bis(trifluoromethanesulfonyl)imide in 0.3 g of acetonitrile and 36 μL of t-butylpyridine (TBP) were added to the chlorobenzene solution, and the contents were mixed. Further, 58 μL of an acetonitrile solution obtained by dissolving 0.11 g of [tris(2-(1H-pyrazol-1-yl)-4-tert-butylpyridine)cobalt(III) tris(bis(trifluoromethylsulfonyl)imide)] in 0.3 g of acetonitrile was mixed thereinto to prepare a coating liquid for a second charge-transporting layer. The coating liquid was applied onto the above-mentioned charge-transporting layer by a spin coating method to form a second charge-transporting layer having a thickness of 80 nm.

### [Formation of First Electrode]

A gold electrode having a thickness of 80 nm and an area of 0.09 cm² was formed on the second charge-transporting layer by a vacuum vapor deposition method. Thus, a photoelectric conversion element was obtained.

### [Short-Circuit Current Density Evaluation]

A power supply (236 model, manufactured by Keithley Instruments) was connected between the electrodes of the photoelectric conversion element. The element was irradiated with constant light through use of a solar simulator (manufactured by Yamashita Denso Corporation) having an intensity of 100 mW/cm², and the density of the generated current was measured. The results are shown in Table 2.

### Comparative Example 1

A photoelectric conversion element was produced and evaluated in the same manner as in Example 1 except that the cyclic conjugated compound was changed to that shown in Table 2 (particle 1 used in Example 1 of Japanese Patent Laid-Open No. 2022-168820). The results are shown in Table 2.

### Examples 2 to 14 and Comparative Example 2

Photoelectric conversion elements are each produced in the same manner as in Example 1 except that the kind of the cyclic conjugated compound, the presence or absence of the resin solution added during the preparation of the charge-transporting layer solution, the kind of the resin solution, the amount of the resin solution, and the presence or absence of the second charge-transporting layer formed are changed. The results are shown in Table 2.

The cyclic conjugated compound is produced in the same manner as in Example 1 by adjusting the dispersion condition (e.g., the number of revolutions) of any cyclic conjugated compound with a sand mill so that the half-width of the peak becomes the value shown in Table 2.

The resin ratio in Table 2 is the content (parts by mass) of the resin in the charge-transporting layer with respect to the content of the cyclic conjugated compound in the charge-transporting layer of 100 parts by mass.

TABLE 2

| | | | | | | | Element |
|---|---|---|---|---|---|---|---|
| | Charge-transporting layer | | | | | | characteristic |
| | | Peak | | | | Second | Short- |
| | | present in | | | Resin | charge- | circuit |
| | | range of | Half- | | ratio | trans- | current |
| | Cyclic conjugated | 2θ of 6.5 | width | | [parts by | porting | density Jsc |
| | compound | to 8.5° [°] | [°] | Kind of resin | mass] | layer | [mA/cm²] |
| Example 1 | Hydroxygallium | 6.9 | 1.1 | Butyral (BM-2) | 10 | Present | 21.2 |
| | phthalocyanine | | | | | | |
| Example 2 | Hydroxygallium | 7.0 | 0.7 | Butyral (BM-2) | 10 | Present | 20.7 |
| | phthalocyanine | | | | | | |
| Example 3 | Hydroxygallium | 6.9 | 1.1 | Butyral (BM-2) | 20 | Present | 20.7 |
| | phthalocyanine | | | | | | |
| Example 4 | Hydroxygallium | 6.9 | 1.1 | Butyral (BM-2) | 5 | Present | 20.8 |
| | phthalocyanine | | | | | | |
| Example 5 | Chlorogallium | 7.3 | 1.1 | Butyral (BM-2) | 10 | Present | 19.8 |
| | phthalocyanine | | | | | | |
| Example 6 | Titanyl | 7.2 | 1.2 | Butyral (BM-2) | 10 | Present | 18.9 |
| | phthalocyanine | | | | | | |
| Example 7 | Aluminum | 7.2 | 1.0 | Butyral (BM-2) | 10 | Present | 18.8 |
| | phthalocyanine | | | | | | |
| Example 8 | Metal-free | 7.3 | 1.1 | Butyral (BM-2) | 10 | Present | 19.0 |
| | phthalocyanine | | | | | | |
| Example 9 | Copper | 7.4 | 1.1 | Butyral (BM-2) | 10 | Present | 17.7 |
| | phthalocyanine | | | | | | |
| Example 10 | 2,3,7,8,12,13,17,18- | 7.9 | 1.4 | Butyral (BM-2) | 10 | Present | 17.0 |
| | Octapheny1-5,10,15,20- | | | | | | |
| | tetraazaporphyrin | | | | | | |
| Example 11 | Hydroxygallium | 6.9 | 1.1 | Butyral (BM-2) | 2 | Present | 20.2 |
| | phthalocyanine | | | | | | |
| Example 12 | Hydroxygallium | 6.9 | 1.1 | Polyacrylic acid | 10 | Present | 20.3 |
| | phthalocyanine | | | (manufactured by | | | |
| | | | | FUJIFILM Wako | | | |
| | | | | Pure Chemical | | | |
| | | | | Corporation) | | | |
| Example 13 | Hydroxygallium | 6.9 | 1.1 | — | — | Present | 19.6 |
| | phthalocyanine | | | | | | |
| Example 14 | Hydroxygallium | 6.9 | 1.1 | Butyral (BM-2) | 10 | Absent | 19.9 |
| | phthalocyanine | | | | | | |
| Comparative | Hydroxygallium | 7.5 | 0.3 | Butyral (BM-2) | 10 | Present | 16.5 |
| Example 1 | phthalocyanine | | | | | | |
| Comparative | Titanyl | 7.3 | 0.3 | Butyral (BM-2) | 10 | Present | 15.6 |
| Example 2 | phthalocyanine | | | | | | |

According to the present disclosure, the photoelectric conversion element having an improved short-circuit current density can be provided.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

1. A photoelectric conversion element comprising:
a first electrode;
a second electrode; and
a photoelectric conversion layer arranged between the first electrode and the second electrode, the photoelectric conversion layer containing a crystal having a perovskite structure,
wherein the photoelectric conversion element comprises, between the photoelectric conversion layer and the first electrode, a charge-transporting layer containing an amorphous body of a cyclic conjugated compound in which a plurality of pyrrole rings are bonded by conjugated bonds, and
wherein, in an X-ray diffraction spectrum of the charge-transporting layer using a CuKα ray, no diffraction peak having a half-width of 0.5° or less is present in a range of a Bragg angle 2θ of 6.5 to 8.5°.

2. The photoelectric conversion element according to claim 1, wherein, in the X-ray diffraction spectrum of the charge-transporting layer using a CuKα ray, a diffraction peak having a half-width of 0.6° or more is present in the range of a Bragg angle 2θ of 6.5 to 8.5°.

3. The photoelectric conversion element according to claim 1, comprising a second charge-transporting layer between the first electrode and the charge-transporting layer.

4. The photoelectric conversion element according to claim 1, wherein the charge-transporting layer contains a resin.

5. The photoelectric conversion element according to claim 4, wherein a content of the resin in the charge-transporting layer is 3 to 30 parts by mass with respect to a content of the cyclic conjugated compound in the charge-transporting layer of 100 parts by mass.

6. The photoelectric conversion element according to claim 1, wherein the cyclic conjugated compound is a phthalocyanine compound.

7. The photoelectric conversion element according to claim 6, wherein the phthalocyanine compound is free of a central metal, or has gallium, aluminum, titanium, or silicon as a central metal.

8. The photoelectric conversion element according to claim 7, wherein the phthalocyanine compound is a gallium phthalocyanine compound.

9. The photoelectric conversion element according to claim 8, wherein the gallium phthalocyanine compound is hydroxygallium phthalocyanine.

10. The photoelectric conversion element according to claim 1, wherein, in the X-ray diffraction spectrum of the charge-transporting layer using a CuKα ray, peaks are present in ranges of a Bragg angle 2θ of 6.8 to 7.10 and 16.4 to 16.8°.

11. A photoelectric conversion apparatus comprising the photoelectric conversion element of claim 1.

12. A moving body comprising:
a photoelectric conversion element,
wherein the photoelectric conversion element comprises:
a first electrode;
a second electrode; and
a photoelectric conversion layer arranged between the first electrode and the second electrode, the photoelectric conversion layer containing a crystal having a perovskite structure,
wherein the photoelectric conversion element comprises, between the photoelectric conversion layer and the first electrode, a charge-transporting layer containing an amorphous body of a cyclic conjugated compound in which a plurality of pyrrole rings are bonded by conjugated bonds, and
wherein, in an X-ray diffraction spectrum of the charge-transporting layer using a CuKα ray, no diffraction peak having a half-width of 0.5° or less is present in a range of a Bragg angle 2θ of 6.5 to 8.5°.

13. A building material comprising:
a photoelectric conversion element,
wherein the photoelectric conversion element comprises:
a first electrode;
a second electrode; and
a photoelectric conversion layer arranged between the first electrode and the second electrode, the photoelectric conversion layer containing a crystal having a perovskite structure,
wherein the photoelectric conversion element comprises, between the photoelectric conversion layer and the first electrode, a charge-transporting layer containing an amorphous body of a cyclic conjugated compound in which a plurality of pyrrole rings are bonded by conjugated bonds, and
wherein, in an X-ray diffraction spectrum of the charge-transporting layer using a CuKα ray, no diffraction peak having a half-width of 0.5° or less is present in a range of a Bragg angle 2θ of 6.5 to 8.5°.
